(19) United States
(12) Patent Application Publication
(10) Pub. No.: US 2012/0108811 A1
(43) Pub. Date: May 3, 2012

(54) PROCESS FOR PREPARING TEMOZOLOMIDE

(75) Inventors: Mei-Jing Lee, Taoyuan (TW); Ching-Peng Wei, Taoyuan (TW)

(73) Assignee: FORMOSA LABORATORIES INC., TAoyuan (TW)

(21) Appl. No.: 12/914,311

(22) Filed: Oct. 28, 2010

(51) Int. Cl. C07D 487/04

(52) U.S. Cl. 544/179

(57) ABSTRACT

This present invention provides an improved process for preparing Temozolomide (TMZ) stable at room temperature for at least 18 months. This present invention also relates to Temozolomide stable at room temperature for at least 18 months.

### FIELD OF THE INVENTION

This present invention relates to an improved process for preparing Temozolomide (TMZ) stable at room temperature for at least 18 months. This present invention also relates to Temozolomide stable at room temperature for at least 18 months.

### DESCRIPTION OF PRIOR ART

Temozolomide is the international non-proprietary name used to identify 8-carbamoyl-3-methyl-imidazo[5,1-d]-1,2,3,5-tetrazin-4(3H)-one (I):

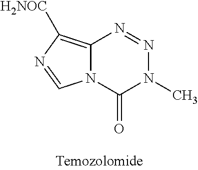

Temozolomide is an antitumor agent indicated for treating patients with malignant glioma such as cancer, breast cancer, refractory anaplastic astrocytoma, i.e., patients at first relapse who have experienced disease progression in malignant glioma, glioblastoma multiform and anaplastic astrocytoma, on a drug regimen containing a nitrosourea and procarbazine.

Temozolomide prepared by the published processes in the art is used to be stored at or below 8° C., usually between about 2 to 8° C. for long-term storage. The process provides Temozolomide stable at room temperature, which is easy for storage, has not been published in the art. Therefore, there remains a need for processes of preparing Temozolomide stable at room temperature.

The preparation of Temozolomide is described in U.S. Pat. No. 7,612,202. This process disclosed a process for preparing Temozolomide base, which comprises contacting Temozolomide hydrochloride with at least one organic acid to produce Temozolomide base and isolating the Temozolomide base. However, the purpose of the organic acid such like acetic acid used in U.S. Pat. No. 7,612,202 is to convert Temozolomide hydrochloride to Temozolomide free base, which is not the same like the present invention. Furthermore, the U.S. Pat. No. 7,612,202 needs special storage and operating condition and equipment for the hydrochloride used in the Temozolomide preparation, which are disadvantageous and costly during the industrial process. Therefore, there remains a need for a process for preparing Temozolomide uses an organic acid which requires no special storage and operating condition.

### BRIEF DESCRIPTION OF THE DRAWINGS

FIG. 1 shows the synthetic scheme of Temozolomide.

### SUMMARY OF THE INVENTION

The present invention provides an improved process for preparing Temozolomide (TMZ) stable at room temperature for at least 18 months. By treating Temozolomide with a mixture of an organic acid, stabilized Temozolomide is obtained. The present invention also relates to Temozolomide stable at room temperature for at least 18 months.

### DETAILED DESCRIPTION OF THE INVENTION

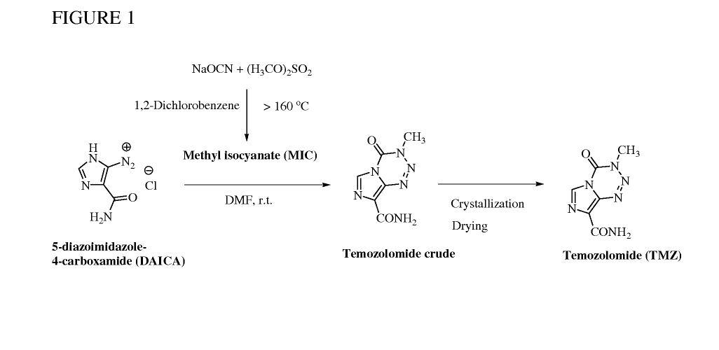

As shown in FIG. 1, the synthesis of Temozolomide (TMZ) starts with 5-diazoimidazole-4-carboxiamide (DAICA). First of all, sodium cyanate is reacted with dimethyl sulfate in 1,2-dichlorobenzene to form methyl isocyanate (CH₃NCO, or MIC) which is in-situ prepared and collected. Second, the in-situ prepared MIC solution is reacted with 5-diazoimidazole-4-carboxiamide in N,N-dimethylformamide (DMF) to form Temozolomide crude. Temozolomide crude is isolated as a solid (wet cake). Then Temozolomide wet cake is crystallized first with acetic acid/water/acetone/charcoal and is then crystallized with acetic acid/water/charcoal to provide the pure Temozolomide.

The present invention relates to a process for the preparation of the stabilized Temozolomide, the process comprising:
(a) adding Temozolomide to a solvent and an organic acid, and charcoal;
(b) heating the mixture to an elevated temperature to form a solution;
(c) filtering the solution to remove the charcoal and obtain a filtrate;
(d) cooling the filtrate to crystallize Temozolomide;
(e) filtering the crystallized Temozolomide; and
(f) drying the wet crystallized Temozolomide under vacuo to obtain the stabilized Temozolomide.

According to one embodiment of the present invention, the solvent is C1˜C6 ketone, C1˜C6 alcohol, C1˜C6 ester, C1˜C6 nitrile, water, or a mixture thereof. The C1˜C6 ketone is preferably acetone, the C1˜C6 alcohol is preferably ethanol and the C1˜C6 nitrile is preferably acetonitrile.

According to another embodiment of the present invention, the organic acid is C1˜C6 aliphatic acid. The aliphatic acid is formic acid, acetic acid, propionic acid, or a mixture thereof, preferably acetic acid.

The present invention also relates to a crystallized Temozolomide prepared by the above-mentioned process for the preparation of the stabilized Temozolomide. The crystallized Temozolomide is stable at room temperature for at least 60 months, preferably 36 months, more preferably 24 months and most preferably 18 months.

According to one embodiment of the present invention, the crystallized Temozolomide has at least 98% detected by a weight-based HPLC assay.

According to another embodiment of the present invention, the crystallized Temozolomide has equal to or less than 0.5% residual solvent, equal to or less than 1.0% total impurity, and no single impurity greater than 0.15% detected by HPLC.

The term “stable” as used herein, refers to the purity of Temozolomide verified by HPLC: Temozolomide >99.0%, 2-aza-hypoxanthine (AHX) <0.10%, 5-aminoimidazole-4-carboxamide hydrochloride (AICA) <0.10% and individual unknown <0.10%.

The advantages of the process of the present invention are the following:

The Temozolomide prepared by the process is stored at room temperature instead of at or below 8° C. Therefore, the Temozolomide of the present invention is easy for storage.

The use of acetic acid in the process instead of hydrochloride avoids the needs for special storage and operating condition and equipment, which are disadvantageous and costly during the industrial process.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Reference is now made to the following examples that, together with the above descriptions, illustrate the invention in a non-limiting fashion. Generally, the nomenclature used herein and the laboratory procedures utilized in the present invention include chemical and analytical techniques with which one skilled in the art is familiar. Unless otherwise defined, technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### EXAMPLES

The examples below are non-limiting and are merely representative of various aspects and features of the present invention.

The manufacturing procedures for Temozolomide were described below. All processes were conducted under a nitrogen atmosphere except for extractions and distillation.

### Example 1

### Preparation of Temozolomide Crude

In reactor B was added 5-diazoimidazole-4-carboxiamide (DAICA, 11 Kg), and N,N-Dimethylformamide (26.4 Kg), the mixture was stirred at room temperature, and waiting for the charging of the in-situ prepared methylisocyanate from reactor A.

In reactor A was added sodium cyanate (27.5 Kg), and 1,2-Dichlorobenzene (DCB, 110 Kg). The mixture was stirred and heated at ambient pressure to distill out DCB (about 12 Kg). After the distillation, dimethyl sulfate (DMS, 52.8 Kg) was slowly added, maintaining the temperature NLT 160° C. during the addition. The methylisocyanate thus generated was vaporized and then condensed into reactor B. After the charging of methylisocyanate into reactor B, the mixture in reactor B was stirred for about three days at room temperature, the reaction progress was monitored by HPLC (DAICA, NMT 1.0%) for the completion of the reaction.

After the reaction, ethyl acetate (124.3 Kg) was charged into reactor B, and the mixture was stirred for at least 30 minutes followed by filtration to give the crude Temozolomide (˜17 kg).

### Example 2

Crystallization of Temozolomide from Acetone, Acetic Acid and Water

In a reactor was added water (358.6 Kg), acetic acid (about 11 Kg), acetone (97.9 Kg), charcoal (2.2 Kg), and crude Temozolomide (˜17 kg). The reaction mixture was heated to around 60° C., followed by filtration in order to remove the charcoal. The filtrate was transferred to another reactor, followed by cooling to allow crystallization. The mixture was stirred at 0±2° C. for at least 1 hour, and then filtered to give the purified Temozolomide (˜12 kg). The purity of the purified Temozolomide was verified by HPLC (Temozolomide >99.0%, 2-aza-hypoxanthine (AHX) <0.15%, 5-aminoimidazole-4-carboxamide hydrochloride (AICA) <0.15%, individual unknown <0.10%).

### Example 3

Crystallization of Temozolomide from Acetic Acid and Water

In a reactor was added water (94.6 Kg), acetic acid (51.6 Kg), charcoal (1.2 kg), and the crude Temozolomide (9 kg). The reaction mixture was heated to around 60° C., followed by filtration in order to remove the charcoal. The filtrate was transferred to another reactor, followed by cooling (maintaining at 36˜40° C. for at least 1 hour, then cooled further to 13˜17° C. for at least 2 hours) to allow crystallization. The crystals were then filtered to give the purified Temozolomide (˜7 kg). The wet product obtained was then dried under vacuum at around 40° C. for about 12 hours to give the final desired stabilized Temozolomide (˜6 kg).

### Example 4

Crystallization of Temozolomide from Acetonitrile, Acetic Acid and Water

To a flask was added water (5 mL), suitable amount of acetic acid (to adjust the pH of the aqueous solution to about 2.5), acetonitril (5 mL), charcoal (100 mg), and the crude Temozolomide (500 mg). The reaction mixture was heated to around 60° C., followed by filtration in order to remove the charcoal. The filtrate was transferred to another flask, followed by cooling to allow crystallization. The mixture was stirred at 0±2° C. for at least 1 hour, and then filtered to give the purified Temozolomide (400 mg). The purity of the purified Temozolomide was verified by HPLC (Temozolomide >99.0%, 2-aza-hypoxanthine (AHX) <0.15%, 5-aminoimidazole-4-carboxamide hydrochloride (AICA) <0.15%, individual unknown <0.10%).

Table 1 showed long-term stability data for Temozolomide prepared by conventional method know in the art. The Temozolomide was stable at temperature 2-8° C.

TABLE 1
Long-term Stability data for Temozolomide
Packing & storage conditions: Sample packed in double polyethylene (PE) bags and in Trilam and kept in a paper drum.

| | | |
|---|---|---|
| Sample qty/time point: 2 g | Temperature: 2-8° C. | Stability study |

| Frequency of analysis: 1, 2, 3, 6, 9, 12, 18, 24, 36, 48 and 60 months | | | | | | | | | duration: 60 |
|---|---|---|---|---|---|---|---|---|---|
| | | | | | | | | | months |
| | | | | Chromatographic purity (HPLC) | | | | | |
| | | | Water | | | Individual | | | |
| | Appearance | Identification | content | | | unknown | Total | Purity | |
| Storage | (White to | (infrared, IR) | (KF), | AHX | AICA | impurity | impurities | (HPLC) | Assay |
| period | off-white | (conform to | NMT | NMT | NMT | NMT | NMT | NLT | (HPLC) |
| (month) | powder) | standard) | 1.00% | 0.15% | 0.15% | 0.10% | 1.00% | 99.0% | (98.0-102.0%) |
| 0 time | White | Conform to | 0.17 | ND | 0.06 | 0.01 | 0.08 | 99.9 | 101.7 |
| | powder | standard | | | | | | | |
| 1 | White | Conform to | 0.36 | ND | 0.02 | 0.02 | 0.03 | 100.0 | 101.4 |
| | crystalline | standard | | | | | | | |
| | powder | | | | | | | | |
| 2 | White | Conform to | 0.20 | ND | 0.04 | 0.01 | 0.06 | 99.9 | 99.8 |
| | powder | standard | | | | | | | |
| 3 | Light beige | Conform to | 0.14 | ND | 0.01 | 0.01 | 0.03 | 100.0 | 99.6 |
| | | standard | | | | | | | |
| 6 | Light beige | Conform to | 0.34 | ND | 0.01 | 0.01 | 0.05 | 99.9 | 99.7 |
| | powder | standard | | | | | | | |
| 9 | Light beige | Conform to | 0.21 | ND | 0.04 | 0.01 | 0.05 | 99.9 | 98.5 |
| | powder | standard | | | | | | | |
| 12 | Light beige | Conform to | 0.11 | ND | 0.06 | 0.02 | 0.09 | 99.8 | 99.2 |
| | powder | standard | | | | | | | |

Trilam: Alluminum foiled bag
AHX: 2-aza-hypoxanthine
AICA: 5-aminoimidazole-4-carboxamide hydrochloride
KF: Karl Fischer titration
ND: not detected.
NMT: No more than.
NLT: No less than.

Table 2 showed long-term stability data for Temozolomide prepared by conventional method know in the art. The Temozolomide was easily to degrade to out of specification at temperature 25 degree ° C.

TABLE 2
Accelerated Stability data for Temozolomide
Packing & storage conditions: Sample packed in double polyethylene (PE) bags and in Trilam and kept in a paper drum.

| | | |
|---|---|---|
| Sample Qty: 6 pieces | Temperature: 25° C. ± 2° C. | Stability study |
| | % Relative humidity: 60% ± 5% | duration: 6 |

| Frequency of analysis: 1, 2, 3 and 6 months | | | | | | | | | months |
|---|---|---|---|---|---|---|---|---|---|
| | | | | Chromatographic purity (HPLC) | | | | | |
| | | | Water | | | Individual | | | |
| | Appearance | Identification | content | | | unknown | Total | Purity | |
| Storage | (White to | (infrared, IR) | (KF), | AHX | AICA | impurity | impurities | (HPLC) | Assay |
| period | off-white | (conform to | NMT | NMT | NMT | NMT | NMT | NLT | (HPLC) |
| (month) | powder) | standard) | 1.00% | 0.15% | 0.15% | 0.10% | 1.00% | 99.0% | (98.0-102.0%) |
| 0 time | White | Conform to | 0.17 | ND | 0.06 | 0.01 | 0.08 | 99.9 | 101.7 |
| | powder | standard | | | | | | | |
| 1 | Off-white | Conform to | 0.25 | ND | 0.24 | 0.01 | 0.25 | 99.8 | 101.7 |
| | crystalline | standard | | | | | | | |
| | powder | | | | | | | | |
| 2 | Off-white | Conform to | 0.24 | ND | 0.64 | 0.01 | 0.67 | 99.3 | 99.4 |
| | powder | standard | | | | | | | |

Trilam: Alluminum foiled bag
AHX: 2-aza-hypoxanthine
AICA: 5-aminoimidazole-4-carboxamide hydrochloride
KF: Karl Fischer titration
ND: not detected.
NMT: No more than.
NLT: No less than.

Table 3 showed long-term stability data for Temozolomide prepared by the process of the present invention. The Temozolomide was stable at temperature 2-8° C. for at least 18 months.

TABLE 3
Long-term Stability data for Temozolomide
Packing & storage conditions: Sample packed in double polyethylene (PE) bags and in Trilam and kept in a paper drum.

| | | |
|---|---|---|
| Sample qty/time point: 2 g | Temperature: 2-8° C. | Stability study |

| Frequency of analysis: 1, 2, 3, 6, 9, 12, 18, 24, 36, 48 and 60 months | | | | | | | | | duration: 60 |
|---|---|---|---|---|---|---|---|---|---|
| | | | | | | | | | months |
| | | | | Chromatographic purity (HPLC) | | | | | |
| | | | Water | | | Individual | | | |
| | Appearance | Identification | content | | | unknown | Total | Purity | |
| Storage | (White to | (infrared, IR) | (KF), | AHX | AICA | impurity | impurities | (HPLC) | Assay |
| period | off-white | (conform to | NMT | NMT | NMT | NMT | NMT | NLT | (HPLC) |
| (month) | powder) | standard) | 1.00% | 0.15% | 0.15% | 0.10% | 1.00% | 99.0% | (98.0-102.0%) |
| 0 time | White | Conform to | 0.09 | ND | 0.01 | ND | 0.01 | 100.0 | 99.7 |
| | powder | standard | | | | | | | |
| 1 | White | Conform to | 0.07 | ND | 0.01 | ND | 0.01 | 100.0 | 98.6 |
| | powder | standard | | | | | | | |
| 2 | White | Conform to | 0.04 | ND | 0.01 | ND | 0.01 | 100.0 | 99.3 |
| | powder | standard | | | | | | | |
| 3 | White | Conform to | 0.09 | ND | 0.09 | ND | 0.09 | 99.7 | 99.6 |
| | powder | standard | | | | | | | |
| 6 | White | Conform to | 0.12 | ND | ND | ND | ND | 100.0 | 99.9 |
| | powder | standard | | | | | | | |
| 9 | White | Conform to | 0.15 | ND | 0.01 | ND | 0.01 | 100.0 | 100.8 |
| | crystalline | standard | | | | | | | |
| | powder | | | | | | | | |
| 12 | Light pink | Conform to | 0.06 | ND | 0.02 | ND | 0.02 | 100.0 | 99.8 |
| | crystalline | standard | | | | | | | |
| | powder | | | | | | | | |
| 18 | White | Conform to | 0.16 | ND | ND | ND | ND | 100.0 | 99.8 |
| | crystalline | standard | | | | | | | |
| | powder | | | | | | | | |

Trilam: Alluminum foiled bag
AHX: 2-aza-hypoxanthine
AICA: 5-aminoimidazole-4-carboxamide hydrochloride
KF: Karl Fischer titration
ND: not detected.
NMT: No more than.
NLT: No less than.

Table 4 showed long-term stability data for Temozolomide prepared by the process of the present invention. The Temozolomide was stable at temperature 25 degree ° C. for at least 18 months.

TABLE 4
Long-term Stability data for Temozolomide
Packing & storage conditions: Sample packed in double polyethylene (PE) bags and in Trilam and kept in a paper drum.

| | | |
|---|---|---|
| Sample qty/time point: 2 g | Temperature: 25° C. ± 2° C. | Stability study |
| | % Relative humidity: 60% ± 5% | duration: 60 |

| Frequency of analysis: 1, 2, 3, 6, 9, 12, 18, 24, 36, 48 and 60 months | | | | | | | | | months |
|---|---|---|---|---|---|---|---|---|---|
| | | | | Chromatographic purity (HPLC) | | | | | |
| | | | Water | | | Individual | | | |
| | Appearance | Identification | content | | | unknown | Total | Purity | |
| Storage | (White to | (infrared, IR) | (KF), | AHX | AICA | impurity | impurities | (HPLC) | Assay |
| period | off-white | (conform to | NMT | NMT | NMT | NMT | NMT | NLT | (HPLC) |
| (month) | powder) | standard) | 1.00% | 0.15% | 0.15% | 0.10% | 1.00% | 99.0% | (98.0-102.0%) |
| 0 time | White | Conform to | 0.09 | ND | 0.01 | ND | 0.01 | 100.0 | 99.7 |
| | powder | standard | | | | | | | |
| 1 | White | Conform to | 0.12 | ND | 0.01 | ND | 0.01 | 100.0 | 100.0 |
| | powder | standard | | | | | | | |
| 2 | White | Conform to | 0.04 | ND | 0.01 | ND | 0.01 | 100.0 | 99.4 |
| | powder | standard | | | | | | | |
| 3 | White | Conform to | 0.11 | 0.01 | 0.11 | ND | 0.11 | 99.7 | 99.4 |
| | powder | standard | | | | | | | |
| 6 | White | Conform to | 0.13 | ND | ND | ND | ND | 100.0 | 99.3 |
| | powder | standard | | | | | | | |
| 9 | White | Conform to | 0.17 | ND | 0.02 | ND | 0.02 | 99.9 | 100.8 |
| | crystalline | standard | | | | | | | |
| | powder | | | | | | | | |
| 12 | Light pink | Conform to | 0.04 | 0.01 | 0.02 | 0.01 | 0.04 | 99.9 | 99.8 |
| | crystalline | standard | | | | | | | |
| | powder | | | | | | | | |
| 18 | White | Conform to | 0.07 | ND | 0.01 | 0.01 | 0.02 | 100.0 | 99.4 |
| | crystalline | standard | | | | | | | |
| | powder | | | | | | | | |

Trilam: Alluminum foiled bag
AHX: 2-aza-hypoxanthine
AICA: 5-aminoimidazole-4-carboxamide hydrochloride
KF: Karl Fischer titration
ND: not detected.
NMT: No more than.
NLT: No less than.

While the invention has been described and exemplified in sufficient detail for those skilled in this art to make and use it, various alternatives, modifications, and improvements should be apparent without departing from the spirit and scope of the invention.

What is claimed is:

1. A process for the preparation of the stabilized Temozolomide, the process comprising:
(a) adding Temozolomide to a solvent and an organic acid, and charcoal;
(b) heating the mixture to an elevated temperature to form a solution;
(c) filtering the solution to remove the charcoal and obtain a filtrate;
(d) cooling the filtrate to crystallize Temozolomide;
(e) filtering the crystallized Temozolomide; and
(f) drying the wet crystallized Temozolomide under vacuo to obtain the stabilized Temozolomide.

2. The process of claim 1, wherein the solvent is C1˜C6 ketone, C1˜C6 alcohol, C1˜C6 ester, C1˜C6 nitrile, water, or a mixture thereof.

3. The process of claim 2, wherein the C1˜C6 ketone is acetone.

4. The process of claim 2, wherein the C1˜C6 alcohol is ethanol.

5. The process of claim 2, wherein the C1˜C6 nitrile is acetonitrile.

6. The process of claim 1, wherein the organic acid is C1˜C6 aliphatic acid.

7. The process of claim 6, wherein the aliphatic acid is formic acid, acetic acid, propionic acid, or a mixture thereof.

8. The process of claim 7, wherein the aliphatic acid is acetic acid.

9. A crystallized Temozolomide prepared by the process of claim 1.

10. The crystallized Temozolomide of claim 9, which is stable at room temperature.

11. The crystallized Temozolomide of claim 10, which is stable at room temperature for at least 18 months.

12. The crystallized Temozolomide of claim 9, which has at least 98% detected by a weight-based HPLC assay.

13. The crystallized Temozolomide of claim 9, which has equal to or less than 0.5% residual solvent, equal to or less than 1.0% total impurity, and no single impurity greater than 0.15% detected by HPLC.
